(19) United States
(12) Patent Application Publication
(10) Pub. No.: US 2008/0200733 A1
(43) Pub. Date: Aug. 21, 2008

(54) Processes For The Preparation Of Purified Solanesol, Solanesyl Bromide & Solanesyl Acetone

(75) Inventors: Abhay Upare, Maharashtra (IN); Ganesh Wagh, Maharashtra (IN); Amit Chavan, Maharashtra (IN); Wazid Sajjad Jafri, Tamilnadu (IN); Sabapathi Selvakumar, Tamilnadu (IN); Mita Roy, Maharashtra (IN)

Correspondence Address: HAMRE, SCHUMANN, MUELLER & LARSON, P.C., P.O. BOX 2902, MINNEAPOLIS, MN, 55402-0902, US

(73) Assignee: NICHOLAS PIRAMAL INDIA LIMITED, Mumbai (IN)

(21) Appl. No.: 11/994,781

(22) PCT Filed: Jun. 21, 2006
(86) PCT No.: PCT/IB2006/052008

§ 371 Date: Mar. 14, 2008

(30) Foreign Application Priority Data
Jul. 6, 2005 (IN) 804/MUM/2005

(51) Int. Cl. C07C 45/85; C07C 45/00; C07C 33/42

(52) U.S. Cl. 568/404; 568/411; 568/849

(57) ABSTRACT

The present invention relates to processes for the preparation of purified solanesol, solanesyl bromide & solanesyl acetone. Solanesyl acetone has the chemical name—all—trans 6,10,14,18,22,26,30,34,38-nonamethyl-5,9,13,17,21,25,29,33,37-triacontanonaen-2-one, of formula 1 and is used for synthesis of coenzyme Q₁₀.

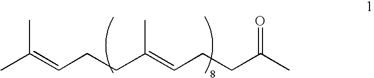

### FIELD OF INVENTION

The present invention relates to process for the preparation of solanesyl acetone, purification of solanesol and process for the preparation of solanesyl bromide. Solanesyl acetone has the chemical name—all—trans 6,10,14,18,22,26,30,34,38-nonamethyl-5,9,13,17,21,25,29,33,37-triacontanonaen-2-one, of formula 1 and is used for synthesis of coenzyme Q₁₀.

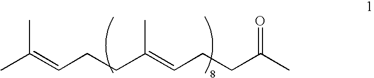

### BACKGROUND AND PRIOR ART

Coenzyme Q₁₀ or COQ₁₀ with the chemical name as 2-[(all-trans)-3, 7,11,15,19,23,27,31,35,39-decamethyl-2,6,10,14,18,22,26,30,34,38-tetracontadecaenyl]-5,6-dimethoxy-3-methyl-1,4-benzoquinone and formula 1A, is present in virtually in every cell in the human body and is known as the “miracle nutrient”. It plays a vital role in maintaining human health and vigor and is involved in mitochondrial processes such as respiration, maintenance of heart muscle strength, enhancement of the immune system, quenching of free radical in the battle against ageing to name a few (“The miracle nutrient coenzyme” Elsevier/North-Holland Biomedical Press, New York, 1986; “Coenzyme Q: Bioechemistry, Bioenergetics, and clinical Applications of Ubiquinone” Wiley, New York, 1985; “Coenzyme Q, Molecular Mechanism in Health and Disease” CRC press). COQ₀ comprises the key raw material for chemical synthesis of CoQ₁₀

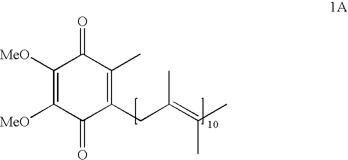

The present invention also provides an improved process for the purification of solanesol & a process for the preparation of solanesyl bromide and solanesyl acetone CoQ₁₀ of the formula 1A comprises of a benzoquinone nucleus attached to a side chain with ten isoprene units.

One of the processes for making CoQ₁₀ is to build the side chain comprising of ten isoprene units and condense with the benzoquinone nucleus. Building up of the side chain is done from solanesol, a naturally occurring alcohol, containing nine isoprene units and having the formula 2

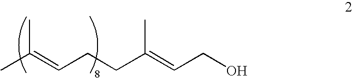

Adding one isoprene unit (five carbons) to solanesol, converts it to decaprenol, of the formula 3, or isodecaprenol of the formula 3i.

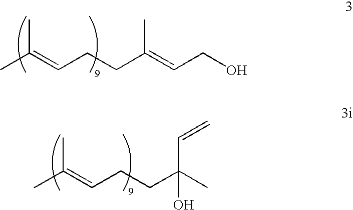

Both the polyprenyl alcohols decaprenol and isodecaprenol contain ten isoprene units and can be used for synthesis of CoQ₁₀. The processes of adding the 10^{th} isoprene unit comprising of five carbon atoms to solanesol of the formula 2 are the following

### Method (I)

Adding a “building block” of five carbon atoms and

### Method (II)

Adding a “building block” of three carbon atoms to form solanesyl acetone of the formula 1 which is then added to a building block of two carbon atoms.

### Method I

The Process of adding “building block” of five carbon atoms to solanesol to form decaprenol 3 has been reported in Jingxi Huagong (2000) 17 (9)549, J. Chem. Soc. Perkin Transc. (1981), 761, Tetrahedron 43 5499(19870)).

This process comprises of two parts, a) Synthesis of the “building block” and b) Condensation of the “building block” with solanesol

Method (Ia) Synthesis of the building block:

For adding five carbon atoms the required “building block” is 4-bromo-3-methyl-2-butenyl acetate of the formula 4.

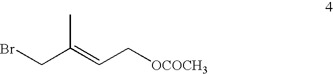

The source of the “building block” is prenyl alcohol of the formula 5 (Scheme-Ia). Prenyl alcohol of the formula 5 is converted by usual method to prenyl acetate of the formula 6.

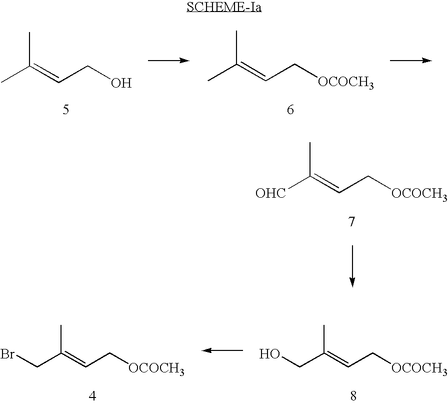

Prenyl acetate of the formula 6 is treated with selenium dioxide (20 times molar excess) in the presence of tert-butyl hydro peroxide (70% solution), in methylene dichloride and is stirred for 50 hours at room temperature. Excess t-butyl hydro peroxide is destroyed with dimethyl sulphide, and the reaction mixture is then neutralized and concentrated to give the crude aldehyde. The aldehyde of the formula 7 is taken in ethanol/methanol and reduced with sodium borohydride. The reaction mixture is quenched in usual way and the crude alcohol 4-hydroxy-3-methyl-2-butenyl acetate of the formula 8 is isolated. The isolated crude alcohol is distilled under vacuum to give the pure product in 30% yield from the acetate of the formula 6. The alcohol is then treated with phosphorus bromide in the presence of pyridine to form 4-bromo-3-methyl-2-butenyl acetate of the formula 4 in 94% yield. Overall yield of synthesizing the building block from prenyl alcohol 5 is only 20%.

In the above process shown in the Scheme-Ia, prenols are known to polymerize, selenium dioxide used in molar excess is toxic and also gives rise to an effluent, the handling of which is cumbersome, the number of steps are many and a very low overall yield. All these above factors make the synthesis not suitable for industrial scale production.

Method (Ib) Condensation of “building block” with solanesol shown in Scheme-Ib

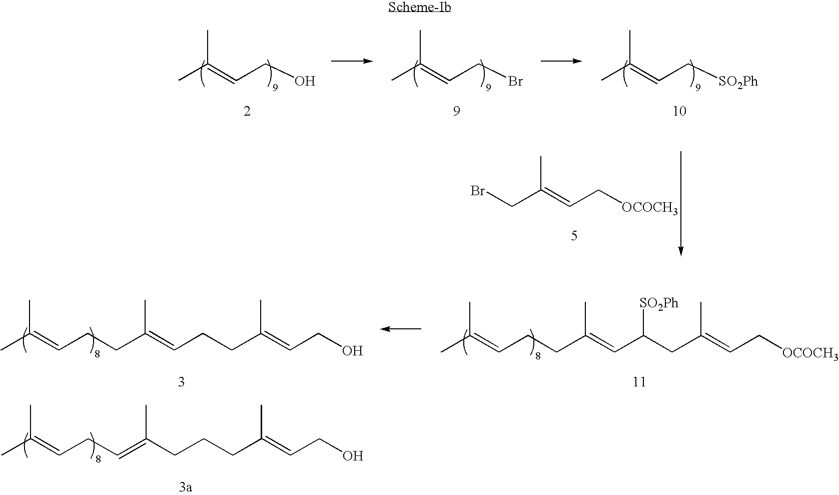

The first step, in the condensation of “solanesol” with the “building block” of formula 4, is synthesis of solanesyl bromide of the formula 9. Solanesyl bromide is made by the reaction of phosphorus tribromide with solanesol of the formula 2. The general methods in literature involves taking solanesol in ether or a mixture of ether and hexane and reacting with phosphorus tribromide in molar ratio of 0.4-1.2 at a temperature in the range of 0-20° C. in the presence of pyridine in 97% yield. The reaction mixture is then quenched in water, the organic layer separated and washed to neutral, dried over sodium sulphate and solvent evaporated to obtain the product in liquid form which is used without further purification (J. Am. Chem. Society (2002), 124, 14282-14283; Jingri Huagong (2000) 17 (9), 549; Recueil de Travas Chimiques de Payas-Bas 113, 153 (1994)).

In the above synthesis of solanesyl bromide of the formula 9 from solanesol uses pyridine, a toxic compound that makes the process not eco friendly. The above process also uses aqueous phase for quenching the reaction mixture and extracts the solanesyl bromide formula 9 formed in a water immiscible solvent.

It has been observed that quenching in aqueous medium results in the formation of an emulsion in the inter phase between the aqueous and organic phases due to which the separation of the organic phase becomes difficult. Such difficulty results in the loss of the final product causing low yield.

Solanesyl bromide of the formula 9 is reacted with sodium benzene sulphinate in N,N-dimethyl formamide to form Solanesol sulphone of the formula 10 in 95% yield.

In the next step, Solanesol sulphone of the formula 10 is treated with 4-bromo-3-methyl-2-butenyl acetate of the formula 5 in the presence of potassium tertiary butoxide or n-butyl lithium. The solvent used is N,N-dimethyl formamide and a mixture of tetrahydrofuran and hexamethyl phosphoramide respectively. The condensed product of the formula 11 thus formed by the above reaction is purified by column chromatography in 59-89% yield. Treating the compound of the formula 11 with sodium amalgam in methanol desulphonates and deprotecting it simultaneously to form decaprenol of the formula 3. Desulphonation with sodium amalgam gives rise to 30% positional isomer, of the formula 3a. The crude decaprenol containing mixture of the compounds of the formulae 3 and 3a is then purified by column chromatography to obtain 3 in 50% yield.

According to the above steps, 30% positional isomer is formed which is an unwanted impurity. This has to be removed by column chromatography, which is not only difficult but also expensive for industrial production. In desulphonation use of sodium amalgum, made from mercury, which is poisonous, is unsafe and therefore not recommended for large-scale production.

Another method of making decaprenol 3 from the condensed product of formula 11 is shown in Scheme-1b (1)

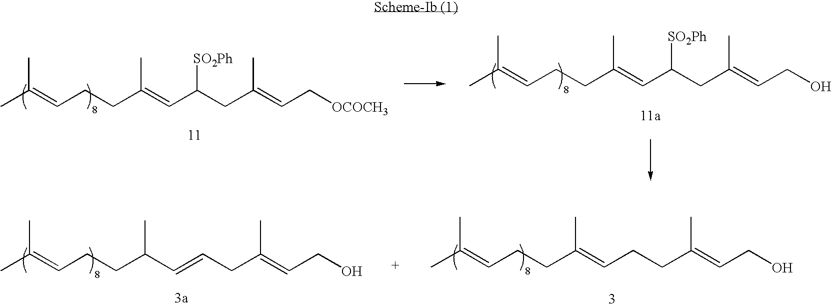

In this method the condensed product of the formula 11 is first deprotected followed by desulphonation to form decaprenol compound of formula 3. Deprotection of compound of formula 11 is carried out by hydrolysis of acetate with potassium hydroxide in 80% methanol, to form alcohol of the formula 11a followed by desulphonation with Li/ethylamine at −78° C. to yield decaprenol of the formula 3 with 10% positional isomer 3a. The crude mixture containing the compounds of the formulae 3 and 3a is purified by column chromatography to obtain the product of the formula 3 in 50-67% yield.

Thus though the positional isomers formed is reduced to 10% from 30% in Scheme Ib, desulphonation by Lithium/ethylamine requires drastic reaction conditions of −78° C. and producing dry ethylamine, and is not suitable for industry.

Preparing decaprenol of the formula 3 from solanesol by the above processes shown in the Schemes Ib and Ib-(1) using a building block of five carbons, results in impurity formation, yield of 40-42%, and involves column chromatography and therefore not an ideal process for scale up. Solanesol being an expensive raw material, such low yield makes the process cost ineffective for industrial production.

### Method II.

Adding a “building block” of three carbon atoms to form solanesyl acetone of the formula 1 which is then added to a building block of two carbon atoms.

The source of “building block” of three carbon atoms and two carbon atoms are ethylacetoacetate of the formula 13, and vinyl magnesium bromide of the formula 14 respectively. Both these compounds are commercially available.

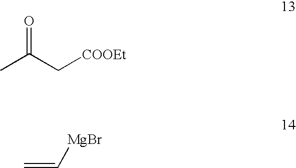

This method comprises of two parts, a) condensation of solanesol with ethyl acetoacetate to form solanesyl acetone Scheme-IIa and (b) condensation of solanesyl acetone with vinyl magnesium bromide to form isodecaprenol, which is easily converted to decaprenol by converting it to acetate followed by hydrolysis (Scheme-IIb).

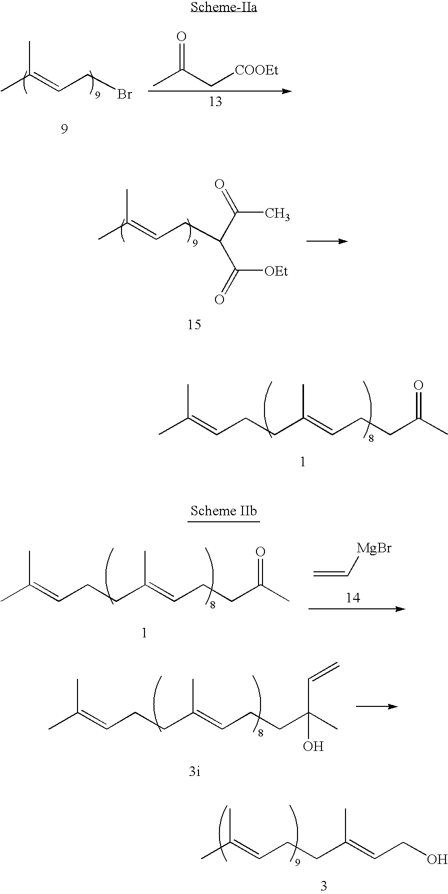

Comparing the Method I and Method II for making decaprenol/isodecaprenol, the method II comprising Scheme-Ia and Scheme-IIb involving solanesyl acetone is simple, with the key raw materials, ethyl acetoacetate and vinyl magnesium bromide, being readily available, less number of steps and no possibility of formation of any positional isomers.

Thus solanesyl acetone would be the preferred intermediate for synthesis of CoQ₁₀ to be used in its synthesis, involving building up of the side chain of ten isoprene units and condensing with the benzoquinone nucleus.

Method of making isodecaprenol and decaprenol from solanesyl acetone using vinyl bromide is reported in Zhurnal Organicheskoi Khimii (1988), 24(6), 1172

Method of preparing CoQ₁₀ from isodecaprenol is given in Chemistry Letters 1597 (1988).

The literature method of making solanesyl acetone is as follows:

The solanesyl acetone is made by treating solanesyl bromide of the formula 9 with ethyl acetoacetate of the formula 13 in the presence of sodium & ethanol at 60° C. to produce solanesyl acetate of the formula 14. The solanesyl acetate is hydrolysed and decarboxylated with 10% sodium hydroxide solution at 60° C. to solanesyl acetone of the formula 1. Recueil de Travas Chimiques de Payas-Bas 113, 153 (1994).

It has been observed that the reaction of solanesyl bromide in such a protic polar solvent and sodium ethoxide gives rise to side reactions such as side chain cyclisation, thereby decreasing the purity of solanesyl acetone produced. The purity of the solanesyl acetone so produced is not more than 65-70% by HPLC. Continuing with the said purity in its further reaction would result in the production of impure COQ₁₀, with a heavy loss in purification, thereby making the process of synthesis of CoQ₁₀ cost ineffective.

Scope of clinical application of coenzyme CoQ₁₀ is becoming wider with its increasing broadband use. An industrially viable, cost effective synthesis of CoQ₁₀ is presently lacking, the present inventors contemplate that an improved process for the preparation of coenzyme CoQ₁₀ can be developed, if the process for the preparation of Solanesyl acetone, an important starting material for the preparation of coenzyme CoQ₁₀, is improved, overcoming the drawbacks of the hitherto known processes.

The major cost-contributing factor for the preparation of CoQ₁₀ is the cost of solanesol. Solanesol is obtained from natural sources namely tobacco and potatoes. The content of Solanesol in tobacco is very less (<2%) and for using it as a starting material for the preparation of CoQ₁₀ it requires a purity of more than 90%. The major problem faced in the industry is the quality of solanesol. Solanesol obtained from commercial source has unwanted residue, and needs purification.

Solanesyl bromide is the first required intermediate in Method II (Scheme-Ia), as discussed above. Therefore, it is essential that the purity of solanesol be maintained in its conversion to solanesyl bromide. Any decrease in purity of solanesyl bromide would affect the subsequent purity of solanesyl acetone of the formula 1. Solanesyl bromide being allylic bromide, its chromatographic purification is ruled out and its low melting point bars the crystallization technique. In short, a clean method of preparation of solanesyl bromide with maximum yield & purity is the need of the hour.

The present invention has been developed on the basis of our findings that Solanesol can be purified by crystallization and its conversion to solanesyl bromide and solanesyl acetone by improved processes.

### OBJECTIVES OF THE PRESENT INVENTION

The main objective of the present invention is to provide an improved process for the preparation of Solanesyl acetone, an important starting material for the preparation of coenzyme CoQ₁₀ overcoming the drawbacks of the hitherto known processes.

Another objective of the present invention is to provide an improved process for the preparation of Solanesyl acetone, which is simple, cost effective and commercially applicable.

Another objective of the present invention is to provide an improved process for the purification of solanesol (purity more than 90%)

Another objective of the present invention is to provide an improved process for the preparation of solanesyl bromide, an important starting material for the preparation of coenzyme CoQ₁₀, circumventing problems of formation of emulsion in the hitherto known processes.

Still another objective of the present invention is to provide an improved process for the preparation of Solanesyl bromide, an important starting material for the preparation of coenzyme COQ₁₀, wherein the yield and purity are over 90%.

### SUMMARY OF INVENTION

The present invention relates to an improved process for the preparation of solanesyl acetone of formula 1, as shown in scheme III below:

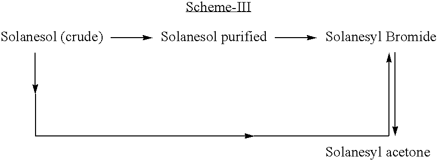

According to a further aspect of the present invention, there is provided an improved process for the purification of solanesol.

According to yet another aspect of the present invention, there is provided an improved process for the preparation of solanesyl bromide of formula 9, which is useful in the preparation of solanesyl acetone.

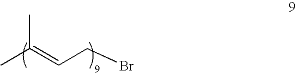

### DETAILED DESCRIPTION OF INVENTION

The process of the present invention is shown in the reaction scheme III shown below

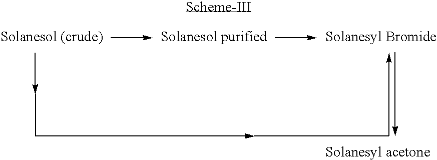

According to an embodiment of the present invention, there is provided an improved process for the synthesis of solanesyl acetone of the formula 1

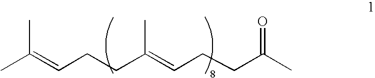

which comprises,
(i) reacting crude or pure solanesol of formula 2

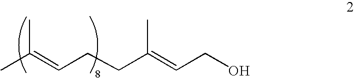

with brominating agent in presence of an acid scavenger selected from alkyl amines;
(ii) quenching the resulting solanesyl bromide of formula 9,

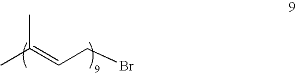

in an aqueous medium to get two phases, namely aqueous and organic phases;
(iii) separating and evaporating the organic phase to isolate the solanesyl bromide of the formula 9;
(iv) reacting solanesyl bromide obtained in (iii) with ethylacetoacetate using a base selected from bulky alkali metal alkoxide base made from tertiary alcohol and mild base like inorganic alkali metal carbonates, in presence of non polar solvent to get the solanesyl ester of the formula 15; and

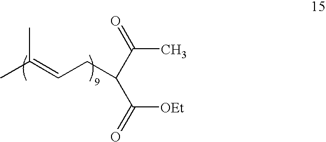

(v) hydrolysing the solanesyl ester of the formula 15 formed in step (iv) by known methods to obtain solanesyl acetone of the formula 1

According to yet another embodiment of the present invention there is provided an improved process for the preparation of solanesyl acetone of the formula 1

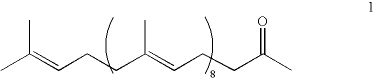

which comprises,
(i) reacting the crude or pure solanesol of formula 2

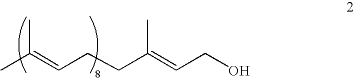

with brominating agent in ether in absence of acid scavenger;
(ii) quenching the resulting solanesyl bromide of the formula 9 in an alcohol to obtain a precipitate which is filtered to obtain a solid;

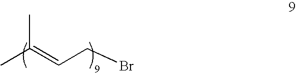

(iii) reacting solanesyl bromide obtained in (ii) with ethylacetoacetate using base selected from bulky alkali metal alkoxide base made from tertiary alcohol, and mild base like inorganic alkali metal carbonates, in presence of non polar solvent to get the solanesyl ester of the formula 15; and

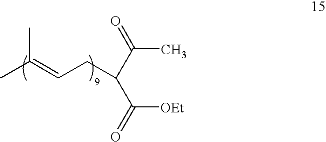

(iv) hydrolysing the solanesyl ester of the formula 15 formed in step (iii) by known methods to get solanesyl acetone of the formula 1

The improvement in the present process of preparation of solanesyl acetone is made by forming the nucleophile of ethyl acetoacetate using a bulky alkali metal alkoxide base made from tertiary alcohol, or a mild base like inorganic alkali metal carbonates, and reacting with solanesyl bromide.

Solanesyl bromide can interact with base forming impurities due to dehalogenation or hydrolysis. Interaction of solanesyl bromide with bulky alkali metal alkoxide base made from tertiary alcohol, is less, because of steric effect, thereby reducing the impurity formation. Inorganic alkali metal carbonates are weak bases and would also interact less effectively with solanesyl bromide.

In prior art an alkyl alkoxide made from primary alcohol that can interact readily with solanesyl bromide were used, thereby increasing the impurity formation.

A nonpolar solvent used in the present invention has negligible solubility of the base thereby decreasing interaction of solanesyl bromide and reducing formation of impurities further, as against prior art where a polar solvent in which the solubility of the base is high is being used.

Solanesyl acetone made by the present invention improves the purity to more than 90% from 65-70% obtained in the prior art.

According to still another embodiment of the present invention there is provided an improved process for the purification of Solanesol of the formula 2, useful in the preparation of solanesyl acetone of the formula 1

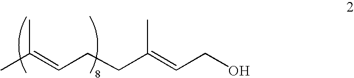

which comprises,
(i) Subjecting crude solanesol to column chromatography using a gradient solvent system selected from non polar, polar and a mixture thereof;
(ii) dissolving the solanesol obtained in step (i) with a polar solvent;
(iii) allowing the resulting solution to settle, and decanting out the supernatant; and
(iv) cooling the supernatant obtained in step (iii) to a temperature in the range of −30° C. to room temperature to get pure (above 90%) solanesol of formula 2

The above method of purification of solanesol uses a combination of column chromatography and crystallization. The crystallization of solanesol in solvent comprising of separating the insoluble by decanting the supernatant from the solution, followed by crystallization is not reported in literature, and therefore novel. The above method improves the purity of solanesol from 75% to about 90%.

According to another embodiment of the present invention there is provided an improved process for the preparation of solanesyl bromide of the formula 9, useful in the preparation of solanesyl acetone of the formula 1

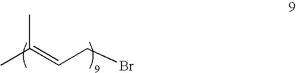

which comprises,
(i) reacting the crude or purified solanesol of formula 2,

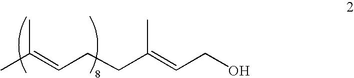

with brominating agent in presence of acid scavenger like alkyl amines;
(ii) quenching the reaction mixture in an aqueous medium to get two phases, namely aqueous and organic phases; separating the organic phase and evaporating to get solanesyl bromide of the formula 9;

Unlike the prior art wherein, pyridine is used as an acid scavenger, the acid scavenger used in the present invention is an alkyl amine. Alkyl amines are non-toxic, environment friendly, economical and therefore commercially viable. Use of an alkyl amine as an acid scavenger, has not been reported in the prior art for making solanesyl bromide and therefore novel.

According to another embodiment of the present invention there is provided an improved process for the preparation of solanesyl bromide of the formula 9, useful in the preparation of solanesyl acetone of the formula 1

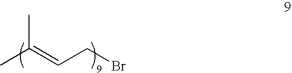

which comprises,
(i) reacting the crude or purified solanesol of formula 2;

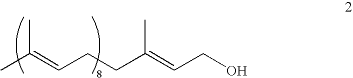

with brominating agent in ether in absence of acid scavenger;
(ii) quenching the reaction mixture in an alcohol to obtain a precipitate which is filtered to obtain solanesyl bromide of formula 9 as a solid;

In the above method of making solanesyl bromide, improvements are effected by, quenching the reaction mixture in alcohol to precipitate out the solid and isolating the solanesyl bromide in solid form by filtering out the solid thereby retaining the coloured impurity in alcohol. The method also avoids the use of aqueous medium thereby circumventing the problem of emulsion, improving the yield and purity of solanesyl bromide to above 95%.

In a preferred embodiment of the invention, column chromatography of crude solanesol may be carried out using silica gel of 60-120 mesh, or 100 to 200 mesh, preferably 60-120 mesh, using a solvent system hexane-ethyl acetate or hexane-dioxane, preferably hexane-ethyl acetate, with loading of silica gel 5 times to 18 times preferably 7-12 times. Elution may be done with 1% ethyl acetate in hexane to 10% ethyl acetate in hexane or 1% dioxane in hexane to 8% dioxane in hexane. Crystallization of column purified solanesol may be done by dissolving in polar solvent like alcohols or ketones like methanol, ethanol, isopropanol, acetone, methyl ethyl ketone, methyl isobutyl ketone etc, preferably alcohol preferably methanol, at temperature in the range of 30-60° C. The solution of solanesol may be allowed to settle and the supernatant solution decanted at a temperature in the range of 10-60° C. preferably at 25-35° C. The supernatant solution of solanesol may be allowed to cool to temperature in the range of −30° C. to 25° C. and the solid may be precipitated out.

The bromination of crude or purified solanesol may be effected employing brominating agents such as phosphorous tribromide, sulphonyl chloride, preferably phosphorous tribromide. The reaction may be carried out in the presence of acid scavenger like alkyl amine such as diethyl amine, triethylamine, diisopropyl amine preferably triethyl amine. The bromination may be carried out in the presence of solvents such as alkanes, ethers, chlorinated hydrocarbons, like hexane, heptane, petroleum ether, diethyl ether, diisopropyl ether. Temperature of reaction may be varied from −10° C. to 25° C. to preferably −5 to −10° C. When the reaction is done in presence of acid scavenger the reaction may be quenched in aqueous medium, and extracted in organic phase.

The bromination of crude or purified solanesol may also be carried out without using an acid scavenger, in presence of solvents such as cyclic ethers like tetrahydrofuran, 1,4-dioxan. When the bromination is effected without an acid scavenger the reaction mixture may be quenched in alcohol like methanol, ethanol or isopropanol preferably methanol thereby avoiding aqueous medium. The volume of methanol may be varied from 5-20 times to that of solanesol preferably 10-15 times. The solid may be precipitated out at a temperature in the range of −20° C. to 20° C.

The solanesyl bromide obtained may be reacted with ethylacetoacetate in hydrocarbon solvent like heptane, hexane, toluene preferably hexane and using a base like alkali metal carbonates like potassium carbonate, sodium carbonate preferably potassium carbonate, or a bulky base like alkali metal alkoxide like sodium tert-butoxide, potassium tert-butoxide, preferably potassium tert-butoxide. The molar ratio of the base with respect to ethylacetoacetate may be varied from 1:0.5 to 1:4 preferably 1:1.0 to 1:2.0.

In the last step the solanesyl ester thus formed may be hydrolyzed in the presence of alkali like sodium hydroxide, potassium hydroxide in aqueous medium or in alcoholic base like ethanolic potassium hydroxide, ethanolic sodium hydroxide or in a solution of alkali in alcohol.

The details of the invention are given in the Examples given below which are given to illustrate the invention only and therefore should not be construed to limit the scope of the invention.

### EXAMPLE 1

### Purification of Solanesol of Formula 3

Solanesol (75% purity) 200 g was impregnated with 250 g silica gel (60-100 mesh size). The column 2.5 ft., 9 inch diameter was packed with silica gel 2.0 Kg. The column was eluted with 1.0 to 6% ethyl acetate in hexane, to obtain 180 g solanesol. Column purified solanesol was taken in methanol (2.5Tit) and heated to 50-55° C. The reaction mixture was transferred to separating funnel at 25-35° C. and allowed to settle. The supernatant was decanted and cooled to 10-15° C. and filtered. Yield 162 g Purity: 90% (HPLC).

### EXAMPLE 2

### Purification of Solanesol of Formula 3

Solanesol (75% purity) 200 g was impregnated with 250 g silica gel (120-300 mesh size). The column 2.5 ft., 9 inch diameter was packed with silica gel 2.0 Kg. The column was eluted with 1.0 to 6% ethyl acetate in hexane to obtain 165 g solanesol. Column purified solanesol (10 g) was taken in acetone and stirred at 25-35° C. The reaction mixture was transferred to separating funnel at 25-35° C. and allowed to settle. The supernatant was decanted and cooled to −30° C. to −25° C. and filtered. Yield: 148 g, Purity: 90%

### EXAMPLE 3

### Preparation of Solanesyl Bromide of Formula 9

Solanesol purified by the process described in Example 1 (44 g), was taken in tetrahydrofuran (132 ml) and cooled to −10° C. Phosphorus tribromide (3 ml) in THF (9 ml) was added dropwise at the same temperature. Reaction was maintained at −10° C. for 2 hrs. Reaction mixture was quenched in methanol (264 ml) at −10° C. to precipitate and filter out solanesyl bromide of formula 9 in form of solid. Yield: 97%, Purity: 92%.

### EXAMPLE 4

### Preparation of Solanesyl Bromide of Formula 9

Solanesol purified by the process described in Example 2, (44 g) was taken in tetrahydrofuran (132 ml) was cooled to −10° C. Phosphorus tribromide (3 ml) in THF (9 ml) was added dropwise at the same temperature. Reaction was maintained at −10° C. for 2 hrs. Reaction mixture was quenched in methanol (440 ml) at 0° C. to precipitate and filter out solanesyl bromide of formula 9 in form of solid. Yield: 92%, Purity: 95%.

### EXAMPLE: 5

### Preparation of Solanesyl Bromide of Formula 9

Solanesol purified by the process described in Example 1, (44 g) was taken in diisopropyl ether (132 ml) and cooled to −10° C. Triethylamine (1.76 g) was added at the same temperature followed by dropwise addition of phosphorus tribromide (3 ml) in diisopropyl ether (9 ml). Reaction was maintained at −10° C. for 2 hrs. Reaction mixture was quenched in water. The organic layer was separated and evaporated to form solanesyl bromide of formula 15. Yield: 94%, Purity: 95%.

### EXAMPLE: 6

### Preparation of Solanesyl Bromide of Formula 9 without Purification of Solanesol

Crude Solanesol (without purification) having the purity of 75%, (44 g) was taken in tetrahydrofuran (132 ml) and cooled to −10° C. Phosphorus tribromide (3 ml) in THF (9 ml) was added dropwise at the same temperature. Reaction was maintained at −10° C. for 2 hrs. Reaction mixture was quenched in methanol (440 ml) at −10° C. to precipitate and filter out solanesyl bromide of formula 9 in form of solid. Yield: 88% Purity: 83%.

### EXAMPLE: 7

### Preparation of Solanesyl Bromide of Formula 9 without Purification of Solanesol

Crude Solanesol (without purification) having the purity of 75%, (44 g) was taken in hexane (132 ml) and cooled to −10° C. Triethylamine (1.76 g) was added at the same temperature, followed by dropwise addition of phosphorus tribromide (3 ml) in hexane (9 ml). Reaction was maintained at −10° C. for 2 hrs. Reaction mixture was quenched in water. The organic layer was separated and evaporated to yield solanesyl bromide compound of formula 9. Yield: 88%; Purity: 83%.

### EXAMPLE 8

### Preparation of Solanesyl Acetone of Formula 1

Potassium carbonate (8.4 g, 1.7 mol) was added to the solution of ethyl acetoacetate (16.4 g) in hexane (250 ml). Solanesyl bromide (25 g) prepared by the process described in Example 3 was added to the reaction mixture and the reaction was continued at room temperature overnight to form solanesyl ester of formula 15. Sodium hydroxide (14.3 g) in water (48 ml) was added to the reaction mixture and the mixture heated to 50° C. overnight. The reaction mixture was quenched in water, and the hexane layer was distilled to obtain solanesyl acetone compound of formula 1 (18.5 g). Yield: 80%, Purity: 76%

### EXAMPLE 9

### Preparation of Solanesyl Acetone of Formula 1

Ethyl acetoacetate (18.3 g) was added to potassium tert-butoxide (7.1 g) in hexane (65 ml) under nitrogen atmosphere. Solanesyl bromide (25 g) prepared by the process described in Example 3, was added to the reaction mixture and the reaction was continued overnight to form compound of formula 15. Reaction mixture was filtered and hexane distilled. The residue was treated with 20% potassium hydroxide solution in isopropanol at 40-45° C. for 2 hrs, quenched in water and the hexane layer was distilled to obtain solanesyl acetone compound of formula 1 (22 g). Yield 91%, Purity 85%.

### EXAMPLE 10

### Preparation of Solanesyl Acetone of Formula 1

Potassium carbonate (8.4 g) was added to the solution of ethyl acetoacetate (16.4 g) in hexane (250 ml). Solanesyl bromide (25 g) prepared by the process described in Example 5 was added to the reaction mixture and the reaction was continued at room temperature overnight to form compound of formula 15. Sodium hydroxide (14.3 g) in water (48 ml) was added to the reaction mixture and the mixture heated to 50° C. overnight. The reaction mixture was quenched in water, and the hexane layer was distilled to obtain solanesyl acetone compound of formula 1 (18.5 g). Yield: 80%, Purity: 76%

### EXAMPLE 11

### Preparation of Solanesyl Acetone of Formula 1

Ethyl acetoacetate (18.3 g) was added to potassium tert-butoxide (7.1 g) in hexane (65 ml) under nitrogen atmosphere. Solanesyl bromide (25 g) prepared by the process described in Example 5, was added to the reaction mixture and the reaction was continued overnight to form compound of formula 15. Reaction mixture was filtered and hexane distilled. The residue was treated with 20% potassium hydroxide solution in isopropanol at 40-45° C. for 2 hrs, quenched in water and the hexane layer was distilled to obtain solanesyl acetone compound of formula 1 (22 g). Yield 91%, Purity 85%.

### EXAMPLE 12

### Preparation of Solanesyl Acetone of Formula 1

Potassium carbonate (8.4 g) was added to the solution of ethyl acetoacetate (16.4 g) in hexane (250 ml). Solanesyl bromide (25 g) prepared by the process described in Example 6 was added to the reaction mixture and the reaction was continued at room temperature overnight to form compound of formula 15. Sodium hydroxide (14.3 g) in water (48 ml) was added to the reaction mixture and the mixture heated to 40-45° C. overnight. The reaction mixture was quenched in water, and the hexane layer was distilled to obtain the solanesyl acetone compound of formula 1 (18.5 g). Yield, 85%, Purity, 80%

### EXAMPLE 13

### Preparation of Solanesyl Acetone of Formula 1

Ethyl acetoacetate (18.3 g) was added to potassium tert-butoxide (7.1 g) in hexane (65 ml) under nitrogen atmosphere. Solanesyl bromide (25 g) prepared by the process described in Example 6, was added to the reaction mixture and the reaction was continued overnight to form compound of formula 15. Reaction mixture was filtered and hexane distilled. The residue was treated with 20% potassium hydroxide solution in isopropanol at 40-45° C. for 2 hrs, quenched in water and the hexane layer was distilled to obtain solanesyl acetone compound of formula 1 (20.5 g). Yield 85%, Purity 80%.

### THE ADVANTAGES OF THE PRESENT INVENTION

1. Purification of Solanesol results in increasing the purity to more than 90%.
2. The process for the preparation of solanesyl bromide is simple and economical and avoids use of toxic reagent.
3. Using purified solanesol, the purity of solanesyl bromide is also enhanced.
4. The process results in making Solanesyl acetone having increased purity of more than 90%.
5. All the processes are robust, simple, economical, environmentally safe and commercially viable.

1-19. (canceled)

20. A process for the preparation of solanesyl acetone of the formula 1 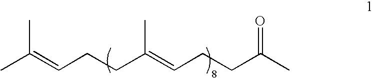
which comprises,
(i) reacting crude or pure solanesol of formula 2 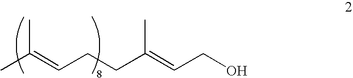
with brominating agent in presence of acid scavenger selected from alkyl amines;
(ii) quenching the resulting solanesyl bromide of formula 9, 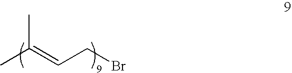
in an aqueous medium to get two phases, namely aqueous and organic phases;
(iii) separating and evaporating the organic phase to isolate the solanesyl bromide of the formula 9;
(iv) reacting solanesyl bromide obtained in (iii) with ethylacetoacetate using base selected from bulky alkali metal alkoxide base made from tertiary alcohol and mild base like inorganic alkali metal carbonates, in presence of non polar solvent to get the solanesyl ester of the formula 15; and 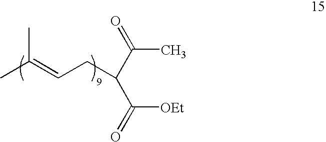
(v) hydrolysing the solanesyl ester of the formula 15 formed in step (iv) by known methods to obtain solanesyl acetone of the formula 1.

21. A process for the preparation of solanesyl acetone of the formula 1 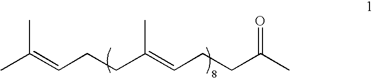
which comprises,
(i) reacting the crude or pure solanesol of formula 2 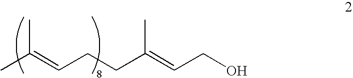
with brominating agent in ether in absence of acid scavenger;
(ii) quenching the resulting solanesyl bromide of the formula 9 in an alcohol to obtain a precipitate which is filtered to obtain a solid; 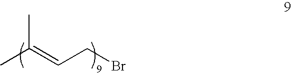
(iii) reacting solanesyl bromide obtained in (ii) with ethylacetoacetate using base selected from bulky alkali metal alkoxide base made from tertiary alcohol, and mild base like inorganic alkali metal carbonates, in presence of non polar solvent to get the solanesyl ester of the formula 15; and 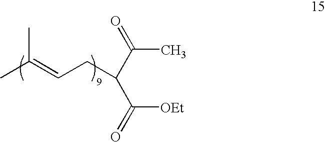
(iv) hydrolysing the solanesyl ester of the formula 15 formed in step (iii) by known methods to get solanesyl acetone of the formula 1

22. A process for the purification of Solanesol of the formula 2, useful in the preparation of solanesyl acetone of the formula 1 as claimed in claims 20 and 21 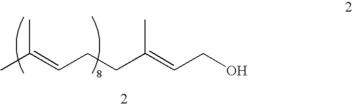
which comprises,
(i) subjecting crude solanesol to column chromatography using a gradient solvent system selected from non polar to polar or a mixture thereof;
(ii) dissolving the solanesol obtained in step (i) with a polar solvent;
(iii) allowing the resulting solution to settle, and decanting out the supernatant; and
(iv) cooling the supernatant obtained in step (c) to a temperature in the range of −30° C. to room temperature to get pure (above 90%) solanesol of formula 2

23. A process for the preparation of solanesyl bromide of the formula 9, 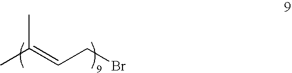
which comprises,
(i) reacting crude or purified solanesol of formula 2, 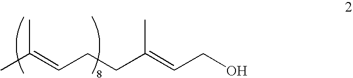
with brominating agent in presence of acid scavenger like alkyl amines;
(ii) quenching the reaction mixture in an aqueous medium to get two phases, namely aqueous and organic phases; separating the organic phase and evaporating to get solanesyl bromide of the formula 9;

24. A process for the preparation of solanesyl bromide of the formula 9, 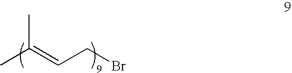
which comprises,
(i) reacting crude or purified solanesol of formula 2; 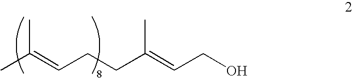
with brominating agent in ether in absence of acid scavenger;
(ii) quenching the reaction mixture in an alcohol to obtain a precipitate which is filtered to obtain solanesyl bromide of formula 9 as a solid.

25. A process as claimed in claim 22 wherein the column chromatography of crude solanesol is carried out using silica gel of 100 to 200 mesh, using a solvent system hexane-dioxane, with loading of silica gel 5 times to 18 times.

26. A process as claimed in claim 22 wherein step (b) is carried out at a temperature in the range 30-60° C., and the polar solvent is selected from alcohols or ketones.

27. A process as claimed in claim 22 wherein in step (c) the warm solution is allowed to settle and the supernatant decanted at a temperature in the range of 10 to 60° C. preferably at 25-35° C.

28. A process as claimed in claim 22 wherein in step (d) the supernatant solution of solanesol is allowed to cool to a temperature in the range of −30° C. to 25° C.

29. A process as claimed in claim 20, wherein the brominating agent is selected from phosphorous tribromide or sulphonyl chloride, in the presence of acid scavenger, and the bromination reaction is carried out in the presence of solvents selected from alkanes, ethers or chlorinated aliphatic hydrocarbons, at a temperature in the range of −10° C. to 25° C. preferably −5 to −10° C.

30. A process as claimed in claim 21, wherein the bromination is effected without using an acid scavenger in solvent of cyclic ethers.

31. A process as claimed in claim 21, wherein the reaction mixture is quenched in alcohol selected from methanol, ethanol and isopropanol, with the volume of methanol varied from 5-20 times to that of solanesol, the solid precipitated out at temperature in the range of −20° C. to 20° C.

32. A process as claimed in claim 20, wherein the reaction of solanesyl bromide with ethylacetoacetate is effected using a weak base.

33. A process as claimed in claim 20, wherein the reaction of solanesyl bromide with ethylacetoacetate is effected using bulky alkali metal alkoxide base selected from sodium tert-butoxide or potassium tert-butoxide, the molar ratio of the base with respect to ethylacetoacetate is varied from 1:0.5 to 1:4.

34. A process as claimed in claims 32 and 33 wherein the reaction is carried out in hydrocarbon solvent selected from heptane or hexane.

35. A process as claimed in claim 25 wherein the column chromatography of crude solanesol is carried out using silica gel of 60-120 mesh.

36. A process as claimed in claim 25 wherein the column chromatography of crude solanesol is carried out using a solvent system hexane-ethyl acetate.

37. A process as claimed in claim 25 wherein the column chromatography of crude solanesol is carried out with loading of silica gel 7-12 times.

38. A process as claimed in claim 26 wherein the polar solvent is selected from methanol, ethanol, isopropanol, acetone, methyl ethyl ketone and methyl isobutyl ketone.

39. A process as claimed in claim 26 wherein the polar solvent is alcohol.

40. A process as claimed in claim 26 wherein the polar solvent is methanol.

41. A process as claimed in claim 29, wherein the brominating agent is phosphorous tribromide.

42. A process as claimed in claim 29, wherein the acid scavenger is a alkyl amine selected from diethyl amine, triethylamine or diisopropyl amine.

43. A process as claimed in claim 29, wherein the bromination reaction is carried out in the presence of solvents selected from hexane, heptane, petroleum ether, diethyl ether, and diisopropyl ether.

44. A process as claimed in claim 30, wherein the bromination is effected without using an acid scavenger in solvent of 1,4 dioxan.

45. A process as claimed in claim 30, wherein the bromination is effected without using an acid scavenger in solvent of tetrahydrofuran.

46. A process as claimed in claim 31, wherein the reaction mixture is quenched in methanol.

47. A process as claimed in claim 31, wherein the reaction mixture is quenched in alcohol with the volume of methanol varied from 10-15 times to that of solanesol.

48. A process as claimed in claim 32, wherein the reaction of solanesyl bromide with ethylacetoacetate is effected using inorganic alkali metal carbonates selected from potassium carbonate and sodium carbonate.

49. A process as claimed in claim 32, wherein the reaction of solanesyl bromide with ethylacetoacetate is effected using potassium carbonate.

50. A process as claimed in claim 33, wherein the reaction of solanesyl bromide with ethylacetoacetate is effected using potassium tert-butoxide.

51. A process as claimed in claim 33, wherein the molar ratio of the base with respect to ethylacetoacetate is from 1:1.0-1:2.

52. A process as claimed in claim 34, wherein the reaction is carried out in a hydrocarbon solvent of hexane.
